(19) United States
(12) Patent Application Publication
(10) Pub. No.: US 2001/0046508 A1
(43) Pub. Date: Nov. 29, 2001

(54) Cosmetic composition

(76) Inventors: Elena shoy, Ridgewood, NY (US); Eduardo shoy, Ridgewood, NY (US)

Correspondence Address: Matthew J. Peirce, Esq., 1550 Starlight Canyon Avenue, Las Vegas, NV, 89123, US

(21) Appl. No.: 09/833,502

(22) Filed: Apr. 11, 2001

Related U.S. Application Data
(60) Provisional application No. 60/195,422, filed on Apr. 10, 2000.

(51) Int. Cl. A61K 35/78; A61K 7/00

(52) U.S. Cl. 424/401; 424/745

(57) ABSTRACT

The present invention concerns that of a new and improved chemical composition for treating and/or removing pimples and acne on a user's face or other body part. The composition would comprise a solution of white rum and rosemary. In order to use composition appropriately, a user would apply the solution to the desired body part three times a day with cotton or cotton applicators.

### BACKGROUND OF THE INVENTION

The present invention concerns that of a new and improved chemical composition for treating and/or removing pimples and acne on a user's face or other body part.

### DESCRIPTION OF THE PRIOR ART

U.S. Pat. No. 5,849,279, issued to Cauwenbergh, discloses a chemical compound for regulating the greasiness of the skin.

U.S. Pat. No. 5,614,201, issued to Slavtcheff et al., discloses a cosmetic composition which includes at least one keratolytic agent and a combination of water-soluble and water-insoluble anti-irritancy agents in a pharmaceutically acceptable carrier.

U.S. Pat. No. 5,248,503, issued to Emanuel-King, discloses the composition of a dietary supplement.

### SUMMARY OF THE INVENTION

The present invention concerns that of a new and improved chemical composition for treating and/or removing pimples and acne on a user's face or other body part. The composition would comprise a solution of white rum and rosemary. In order to use composition appropriately, a user would apply the solution to the desired body part three times a day with cotton or cotton applicators.

There has thus been outlined, rather broadly, the more important features of a chemical composition for treating and/or removing pimples and acne in order that the detailed description thereof that follows may be better understood and in order that the present contribution to the art may be better appreciated. There are, of course, additional features of the chemical composition for treating and/or removing pimples and acne that will be described hereinafter and which will form the subject matter of the claims appended hereto.

In this respect, before explaining at least one embodiment of the chemical composition for treating and/or removing pimples and acne in detail, it is to be understood that the chemical composition for treating and/or removing pimples and acne is not limited in its application to the details of construction and to the arrangements of the components set forth in the following description or illustrated in the drawings. The chemical composition for treating and/or removing pimples and acne is capable of other embodiments and being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present chemical composition for treating and/or removing pimples and acne. It is important, therefore, that the claims be regard as including such equivalent constructions insofar as they do not depart from the spirit and scope of the present invention.

It is therefore an object of the present invention to provide a chemical composition for treating and/or removing pimples and acne which has all of the advantages of the prior art and none of the disadvantages.

It is another object of the present invention to provide a chemical composition for treating and/or removing pimples and acne which may be easily and efficiently manufactured and marketed.

It is yet another object of the present invention to provide a chemical composition for treating and/or removing pimples and acne which is economically affordable and available for the public.

Other objects, features and advantages of the present invention will become more readily apparent from the following detailed description of the preferred embodiment when considered with the attached drawings and appended claims.

### DESCRIPTION OF THE PREFERRED EMBODIMENT

Applicant teaches a new and improved cosmetic composition in liquid form, for application to a user's skin. The cosmetic composition would be designed for treating and/or removing pimples and acne on a user's face or other body parts. The cosmetic composition would be in solution and comprise two ingredients, which would be a volume of rum and a portion of rosemary.

The rum to be used with the present invention would preferably be white rum. The white rum can either be a generic brand or can be a trademarked brand, such as Captain Morgan's, Bacardi, or other well-known brands.

The rosemary to be used would be of a commercial variant. The inventor has discovered that a few (two to three) teaspoons of rosemary, mixed with a small amount of white rum, will create the composition that can be used by a user to prevent and eliminate pimples and acne from the face or other desired body parts.

The cosmetic composition would be applied with a cotton applicator, which can be used to apply the composition of the present invention to a user's face or other specified body part. A cotton ball would preferably be used if a user would prefer an application over a substantial part of a particular body part, while a cotton swab would preferably be used if a user would want to pinpoint the application of the composition of the present invention to a particular small area of their face or other particular body part.

What I claim as my invention is:

1. A cosmetic chemical composition for treating and/or removing pimples and acne comprising:
(a) an amount of white rum, and
(b) a few teaspoons of rosemary.

2. A cosmetic chemical composition for treating and/or removing pimples and acne according to claim 1 wherein the white rum would be a generic brand name.

3. A cosmetic chemical composition for treating and/or removing pimples and acne according to claim 1 wherein the white rum would be a trademarked brand name.
